(19) United States
(12) Patent Application Publication
(10) Pub. No.: US 2021/0024386 A1
(43) Pub. Date: Jan. 28, 2021

(54) Faucet capable of supplying ozone water

(71) Applicant: Guangzhou Seagull Kitchen And Bath Products Co.,Ltd., Guangzhou (CN)

(72) Inventors: Yuanhung YANG, New Taipei (CN); Andy OU, Xinyi (CN)

(21) Appl. No.: 17/015,114

(22) Filed: Sep. 9, 2020

(30) Foreign Application Priority Data
Jul. 9, 2019 (CN) 201910612880.0

(51) Int. Cl. C02F 1/68; B01F 3/04; B01F 5/04

(52) U.S. Cl. CPC C02F 1/685; B01F 3/0446; B01F 3/04829; B01F 15/00409; B01F 2003/04886; B01F 2215/008; B01F 5/0428

(57) ABSTRACT

The invention discloses a faucet capable of discharging ozone water, which includes a faucet body; an ozone generator; a water-gas mixer which includes a water inlet and a water outlet respectively located at two ends, wherein the water-gas mixer is arranged on a water outlet pipeline of the faucet body through the water inlet and the water outlet, an inner diameter reducing portion is formed in the water-gas mixer, a gas inlet is arranged at a position of a side wall of the water-gas mixer corresponding to the inner diameter reducing portion, and the gas inlet is communicated with the ozone generator; and a sensor arranged on the faucet body and used to receive an inductive signal to switch on or switch off the ozone generator.

This application claims priority to Chinese Patent Application Ser. No. CN201910612880.0 filed 9 Jul. 2019.

### TECHNICAL FIELD

The present invention relates to a faucet. More particularly, the present invention relates to a faucet capable of discharging ozone water.

### BACKGROUND ART

The most suitable way to disinfect and sterilize with ozone is to dissolve the ozone in water to form so-called “ozone water”. The “ozone water” has a particularly obvious effect on sterilization and disinfection of clothes, bags and the like, has functions of sterilization and disinfection, preservation and fresh-keeping, and peculiar smell removal on fruits, vegetables and meat, and also has a function of degradation of phosphorus-containing pesticides left on surfaces of fruits and vegetables. However, there is no ozone disinfection device which is suitable for families and can use the ozone water as needed currently.

### SUMMARY

One objective of the present invention is to provide a faucet capable of discharging ozone water, in which the ozone generator can be switched on to obtain ozone as needed, so that the ozone water can be used as needed.

In order to achieve these objectives and other advantages according to the present invention, a faucet capable of discharging ozone water is provided, which includes:

a faucet body;

an ozone generator;

a water-gas mixer which includes a water inlet and a water outlet respectively located at two ends, wherein the water-gas mixer is arranged on a water outlet pipeline of the faucet body through the water inlet and the water outlet, an inner diameter reducing portion is formed in the water-gas mixer, a gas inlet is arranged at a position of a side wall of the water-gas mixer corresponding to the inner diameter reducing portion, and the gas inlet is communicated with the ozone generator; and

a sensor arranged on the faucet body and used to receive an inductive signal to switch on or switch off the ozone generator.

Preferably, according to the faucet capable of discharging ozone water, the sensor includes a touch sensor, and when a pressing time detected by the touch sensor is greater than a set threshold, the ozone generator is switched on; and when a pressing time detected by the touch sensor again is greater than the set threshold, the ozone generator is switched off.

Preferably, according to the faucet capable of discharging ozone water, the sensor further includes an infrared sensor, and when the infrared sensor detects that an infrared signal changes, the ozone generator is switched on.

Preferably, according to the faucet capable of discharging ozone water, the faucet body includes a valve and a faucet nozzle, and the water-gas mixer is arranged on a water outlet pipeline between a water outlet of a valve spool and the faucet nozzle.

Preferably, the faucet capable of discharging ozone water further includes:

an indicator lamp arranged on the faucet body, wherein the indicator lamp flashes when the ozone generator is switched on.

Preferably, the faucet capable of discharging ozone water further includes:

a hot water inlet pipeline and a cold water inlet pipeline, wherein the hot water inlet pipeline and the cold water inlet pipeline are respectively used to supply hot water and cold water to the faucet body.

Preferably, according to the faucet capable of discharging ozone water, the valve is a mechanical valve or an electric control valve.

Preferably, according to the faucet capable of discharging ozone water, the ozone generator includes an air inlet and an ozone outlet, the ozone outlet is communicated with the gas inlet, a sound sensor is arranged at the air inlet, and when sound of air inflow is not detected by the sound sensor, the ozone generator is switched off.

Preferably, according to the faucet capable of discharging ozone water, the water-gas mixer is venturi tube-shaped.

Preferably, the faucet capable of discharging ozone water further includes:

a transformer used to supply power to the ozone generator.

The present invention includes at least the following beneficial effects.

The present invention prevents the ozone water from being discharged as soon as the faucet is turned on, thus preventing accidental drinking easily caused by long-time discharge of the ozone water, and preventing a defect that a service life of the ozone generator is affected by long-time operation at the same time. The present invention uses an inductive switch arranged on the faucet to trigger and switch on the ozone generator when the ozone water is needed, and then the ozone water is discharged from the faucet nozzle, thus achieving the purpose of reasonably using the ozone water as needed.

Other advantages, objectives and features of the present invention will be partially reflected by the following description, and will be partially understood by those skilled in the art through study and practice of the present invention.

### BRIEF DESCRIPTION OF THE DRAWINGS

FIG. 1 is an overall schematic diagram of the present invention;

FIG. 2 is a schematic diagram of a capacitive touch key and an indicator lamp of the present invention;

FIG. 3 is a principle diagram of a water-gas mixer of the present invention;

FIG. 4 is a working principle diagram of the present invention with mechanical valve spool control; and

FIG. 5 is a working principle diagram of the present invention with electromagnetic valve control.

### DETAILED DESCRIPTION

The present invention is further described in detail hereinafter with reference to the accompanying drawings, so that those skilled in the art can implement according to the specification.

It should be understood that the terms such as “have”, “contain” and “comprise” used herein do not exclude the presence or addition of one or more other elements or combinations thereof.

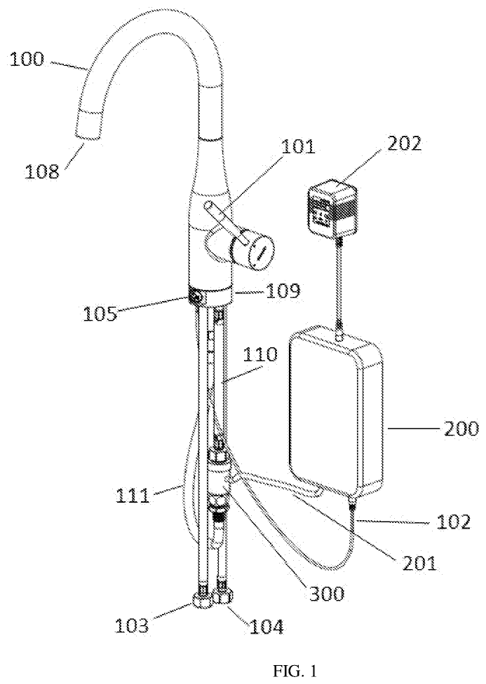

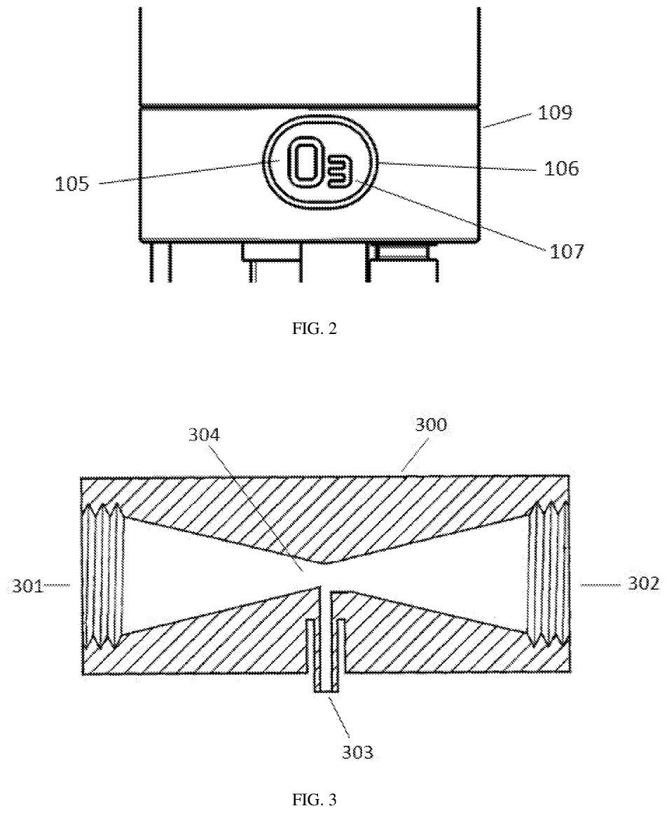

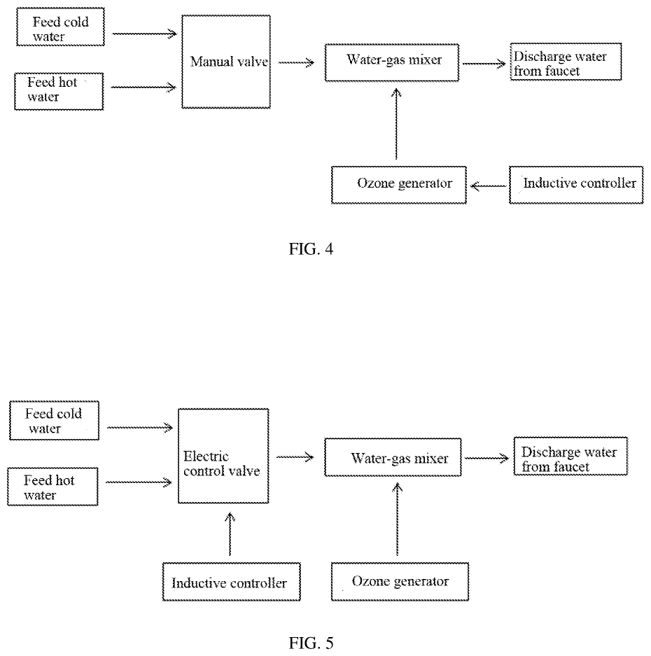

In one technical solution, as shown in FIG. 1 and FIG. 2, a faucet capable of discharging ozone water includes:

a faucet body 100;

an ozone generator 200;

a water-gas mixer 300 which includes a water inlet 301 and a water outlet 302 respectively located at two ends, wherein the water-gas mixer is arranged on a water outlet pipeline of the faucet body through the water inlet and the water outlet, an inner diameter reducing portion 304 is formed in the water-gas mixer, a gas inlet 303 is arranged at a position of a side wall of the water-gas mixer corresponding to the inner diameter reducing portion, and the gas inlet is communicated with the ozone generator; and

a sensor 105 arranged on the faucet body and used to receive an inductive signal to switch on or switch off the ozone generator.

In the above technical solution, the faucet body and the ozone generator may refer to the prior art, and the faucet may include various faucets, such as an electric control faucet or a mechanical faucet, and the ozone generator is preferably a small-sized generator. The water-gas mixer is used to mix ozone and water, and the water-gas mixer is internally provided with a water flow channel, which is communicated with the water outlet pipeline. The water flow channel is internally provided with the inner diameter reducing portion, so that when water flows through the water flow channel, a negative pressure is generated at the inner diameter reducing portion. The inner diameter reducing portion is provided with the gas inlet, which is communicated with the ozone generator. When the ozone generator is switched on, the negative pressure generated at the inner diameter reducing portion may suck ozone into the water-gas mixer, and mix the ozone with water to obtain the ozone water. The faucet body has a base 109, which is provided with the sensor 105, and 102 is a signal line of the sensor. The ozone generator is switched on and off through the inductive signal. The sensor may be a contact sensor or a non-contact sensor. When the inductive signal is not received, the technical solution may be used as a common faucet, and when the inductive signal is received, the technical solution provides the ozone water. It can be seen that the technical solution does not need to additionally provide a separate pipeline for the ozone water, and the ozone water shares the water outlet pipeline of the faucet body, so that a structure is relatively simple. After the valve of the faucet is closed, the ozone may not enter the water-gas mixer again, and the ozone water may no longer be generated. When the ozone water is needed, the ozone generator is switched on by the sensor, and the ozone water is discharged from the faucet nozzle, thus achieving the purpose of reasonably using the ozone water as needed.

In another technical solution, according to the faucet capable of discharging ozone water, the sensor includes a touch sensor, and when a pressing time detected by the touch sensor is greater than a set threshold, the ozone generator is switched on; and when a pressing time detected by the touch sensor again is greater than the set threshold, the ozone generator is switched off. A form of the sensor is provided herein, which means that when a touch induction signal of a user is received, the ozone generator is switched on, and when a duration of the touch sensor exceeds a threshold, the ozone generator is switched off.

In another technical solution, according to the faucet capable of discharging ozone water, the sensor further includes an infrared sensor, and when the infrared sensor detects that an infrared signal changes, includes ozone generator is switched on. A second form of the sensor is provided herein, when the user changes the infrared signal with physical movements, the ozone generator 200 is switched on without touching the sensor, thus keeping a surface of the sensor clean.

In another technical solution, according to the faucet capable of discharging ozone water, the faucet body includes a valve and a faucet nozzle 108, and the water-gas mixer is arranged on a water outlet pipeline between a water outlet of a valve spool and the faucet nozzle. A structure of the faucet body and an arrangement mode of the water-gas mixer are provided herein, so that whether the ozone water is supplied may be selected by opening and closing the valve. Specifically, as shown in FIG. 1, 110 is a valve output hose and 108 is a drawing hose, both of which are respectively connected with the water inlet 301 and the water outlet 302 of the water-gas mixer, and 201 is a silicone hose for connecting the ozone generator and the gas inlet.

In another technical solution, the faucet capable of discharging ozone water further includes:

an indicator lamp 106 arranged on the faucet body, wherein the indicator lamp flashes when the ozone generator is switched on. Here, the indicator lamp flashes to remind the user that the faucet supplies the ozone water, thus reducing accidental drinking of the user. 107 is an ozone character indication and is used to remind the user to pay attention.

In another technical solution, the faucet capable of discharging ozone water further includes:

a hot water inlet pipeline and a cold water inlet pipeline, wherein the hot water inlet pipeline 103 and the cold water inlet pipeline 104 are respectively used to supply hot water and cold water to the faucet body. Some auxiliary structures of the faucet body are provided herein to provide the user with basic hot and cold water functions.

In another technical solution, according to the faucet capable of discharging ozone water, the valve is a mechanical valve 101 or an electric control valve. Two control forms of valves are provided herein, which are namely mechanical valve spool control and electromagnetic valve control.

In another technical solution, according to the faucet capable of discharging ozone water, the water-gas mixer is venturi tube-shaped. A specific form of the water-gas mixer is provided herein, which is convenient for sucking the ozone.

In another technical solution, according to the faucet capable of discharging ozone water, the ozone generator includes an air inlet and an ozone outlet, the ozone outlet is communicated with the gas inlet, a sound sensor is arranged at the air inlet, and when sound of air inflow is not detected by the sound sensor, the ozone generator is switched off. A way to automatically switching off the ozone generator is provided herein, which communicates the ozone generator with the gas inlet. A venturi effect of the water-gas mixer is used to help the ozone generator to suck air, and oxygen is converted into the ozone in the ozone generator and is further sucked into the water-gas mixer. The air inlet is provided with a sound sensor. When the sound of air inflow is detected, no operation is performed, and when the sound of air inflow is not detected, the ozone generator is switched off. The sound sensor may pre-store characteristics of the sound of air inflow for comparison with an actually detected sound.

In another technical solution, the faucet capable of discharging ozone water further includes:

a transformer 202 used to supply power to the ozone generator. The ozone generator usually includes a high-voltage electrode, and the transformer is used to provide the ozone generator with a voltage higher than the mains supply.

A number of devices and a processing scale described herein are used to simplify the description of the present invention. The application, modification and variation of the faucet capable of discharging ozone water according to the present invention are apparent to those skilled in the art.

Although the embodiments of the present invention have been disclosed above, the present invention is not limited to the applications listed in the description and the embodiments. The present invention can be applied to various fields suitable for the present invention absolutely, and other modifications can be easily realized by those skilled in the art. Therefore, the present invention is not limited to the specific details and the illustrations shown and described herein without departing from the general concepts defined by the claims and equivalent scopes.

What is claimed is:

1. A faucet capable of discharging ozone water, comprising:
a faucet body;
an ozone generator;
a water-gas mixer which comprises a water inlet and a water outlet respectively located at two ends, wherein the water-gas mixer is arranged on a water outlet pipeline of the faucet body through the water inlet and the water outlet, an inner diameter reducing portion is formed in the water-gas mixer, a gas inlet is arranged at a position of a side wall of the water-gas mixer corresponding to the inner diameter reducing portion, and the gas inlet is communicated with the ozone generator; and
a sensor arranged on the faucet body and used to receive an inductive signal to switch on or switch off the ozone generator.

2. The faucet capable of discharging ozone water according to claim 1, wherein the sensor comprises a touch sensor, and when a pressing time detected by the touch sensor is greater than a set threshold, the ozone generator is switched on; and when a pressing time detected by the touch sensor again is greater than the set threshold, the ozone generator is switched off.

3. The faucet capable of discharging ozone water according to claim 2, wherein the sensor further comprises an infrared sensor, and when the infrared sensor detects that an infrared signal changes, the ozone generator is switched on.

4. The faucet capable of discharging ozone water according to claim 1, wherein the faucet body comprises a valve and a faucet nozzle, and the water-gas mixer is arranged on a water outlet pipeline between a water outlet of a valve spool and the faucet nozzle.

5. The faucet capable of discharging ozone water according to claim 1, further comprising:
an indicator lamp arranged on the faucet body, wherein the indicator lamp flashes when the ozone generator is switched on.

6. The faucet capable of discharging ozone water according to claim 1, further comprising:
a hot water inlet pipeline and a cold water inlet pipeline, wherein the hot water inlet pipeline and the cold water inlet pipeline are respectively used to supply hot water and cold water to the faucet body.

7. The faucet capable of discharging ozone water according to claim 4, wherein the valve is a mechanical valve or an electric control valve.

8. The faucet capable of discharging ozone water according to claim 1, wherein the water-gas mixer is venturi tube-shaped.

9. The faucet capable of discharging ozone water according to claim 1, wherein the ozone generator comprises an air inlet and an ozone outlet, the ozone outlet is communicated with the gas inlet, a sound sensor is arranged at the air inlet, and when sound of air inflow is not detected by the sound sensor, the ozone generator is switched off.

10. The faucet capable of discharging ozone water according to claim 1, further comprising:
a transformer used to supply power to the ozone generator.
